(19) United States
(12) Patent Application Publication
(10) Pub. No.: US 2021/0121162 A1
(43) Pub. Date: Apr. 29, 2021

(54) MEDICAL APPARATUS WITH SEGMENTED BENDABLE SECTIONS

(71) Applicant: Canon U.S.A., Inc., Melville, NY (US)

(72) Inventors: Brian Ninni, Woburn, MA (US); Takahisa Kato, Brookline, MA (US); Benedict Shia, Needham, MA (US); Showna Chang, Arlington, MA (US); Matthew Michael Kincaid, Medford, MA (US)

(21) Appl. No.: 17/077,480

(22) Filed: Oct. 22, 2020

Related U.S. Application Data
(60) Provisional application No. 62/925,101, filed on Oct. 23, 2019.

(51) Int. Cl. A61B 10/02; A61B 34/20; A61B 90/00; A61B 34/00

(52) U.S. Cl. CPC A61B 10/0233; A61B 34/20; A61B 6/032; A61B 34/71; A61B 2034/2051; A61B 90/39

(57) ABSTRACT

An articulated medical device having a hollow core, capable of large degrees of maneuverability through small spaces of a patient to reach a target with minimal invasiveness, and once the medical device has reached the target, allowing a medical tool to be guided through the hollow chamber for facilitating medical procedures, including endoscopes, cameras, and bendable medical devices, at the target.

### CROSS REFERENCE TO RELATED PATENT APPLICATIONS

This application claims priority from U.S. Provisional Patent Application No. 62/925,101 filed on Oct. 23, 2019 in the United States Patent and Trademark Office, the disclosure of which is incorporated herein, in its entirety, by reference.

### FIELD OF THE DISCLOSURE

The present disclosure relates generally to apparatus and methods for medical application. More particularly, the subject disclosure is directed to an articulated medical device having a hollow chamber, wherein the device is capable of maneuvering within a patient to reach a desired target, and allowing a medical tool to be guided through the hollow chamber, allowing for medical procedures at the target. The medical tool may include an endoscope, camera, bendable medical devices or other medical implements.

### BACKGROUND OF THE DISCLOSURE

Bendable medical instruments such as endoscopic surgical instruments and bendable medical devices are well known and continue to gain acceptance in the medical field. The bendable medical instrument generally includes a flexible body commonly referred to as a sleeves or sheaths. One or more tool channels extend along (typically inside) the flexible body to allow access to a target located at a distal end of the body.

The instrument is intended to provide flexible access within a patient, with at least one curve or more leading to the intended target, while retaining torsional and longitudinal rigidity so that a physician can control the tool located at the distal end of the medical instrument by maneuvering the proximal end of the instrument.

Recently, to enhance maneuverability of the distal end of the instrument, robotized instruments that control distal portions have emerged. In those robotized instruments, to create curves locally at the distal portion by robotics, different techniques have been disclosed.

By way of example, United States patent publication number 2018/0243900 provides a multi-sectional articulated bendable medical device having bending sections where all sections are comprised of multiple individual nodes/guide rings. The prior patents involve either a fully “skeleton” section, made up of many individual guide rings, or a single piece section made of multi-guide hole tubing.

In either case, the art provides multiple conduits to retain the shape of the proximal part, while the driving tendons are bending the distal part in the medical instruments. The multiple conduits would be controlled selectively in a binary way by constraining or unconstraining the proximal ends of the conduits. By selecting the constrained conduits, the bendable medical device can change the length of bending distal segment by changing the stiffness of the bendable medical device based on the area where the conduits deploy.

However, the existing medical instrument has several shortcomings, thereby severely limiting their use and efficacy. Namely, column stiffness necessary to propel the instrument through difficult areas without collapsing has been particularly challenging, especially when minimizing the overall diameter of the instrument. In addition, the use of a medical instrument having a single-structure proximal section lacks the flexibility to maneuver through tight bends. As the length of the section plays a role as well, a short bending section for a single structure proximal section will require a much larger force to bend the instrument to the desired output angle than what is feasible. A short length skeleton structure can provide optimal maneuverability, but the preceding passive section needs to be stiff to allow the skeleton structure to bend properly, as well as endure the forces placed upon them when bent, which makes it difficult to travel through tortuous paths.

Finally, the implementation of a proximal section with both the skeleton structure and multi-guide hole tubing is not robust enough to convey the pushing force required to navigate through the lung airways. While the skeleton part of the proximal section can navigate through certain bifurcations of the lungs, the multi-guide hole tubing following the skeleton structure, tends to fail to turn the same bifurcation, and instead start to buckle and prolapse into other opening of the bifurcation. When this happens, the robot system cannot convey the pushing force to the distal end of the bendable medical device, so the prolapse limits reachability of the bendable medical device to nodules in peripheral regions of the lung. Further exacerbating this problem, the bendable medical device prolapse often leads to permanent damage rendering the medical instrument useless.

Some attempts to cure this deficiency have included adopting longer bending sections, however, these attempts suffer from requiring a large displacement when bending, making it difficult to control the bendable medical device which is potentially harmful to a patient.

The length difference between the distal section and proximal section results in an inaccurate path following and large tip motion during progression of the apparatus into a patient. This tip dislocation causes the user to attempt to adjust the tip to re-align with the original trajectory. However, this motion should not be necessary for the bendable medical device to follow the path of advancement into the patient, so as the instrument progresses, the middle and proximal sections will be bending in directions that are not along the desired path. This process will move the tip in an undesired direction, and the derailment cycle will continue, exaggerated with each step. The stiffness of the proximal section results in poor and inconsistent advancement of the medical instrument, since the bending output does not match the output of the middle of proximal section, meaning it will not reach the same angle as it should, and potentially harm the patient.

### SUMMARY

Thus, to address such exemplary needs in the industry, the presently disclosed apparatus teaches a medical apparatus comprising: a bendable body having a hollow chamber extending the length of the bendable body; a first bendable section; an intermediate bendable section; at least two guide rings disposed in the first bendable section and spaced a distance from one another to create a cavity; at least two guide holes in the bendable body, the guide holes extending the length of the bendable body and parallel with the hollow chamber; and at least one driving wire slideably situated in at least one of the at least two guide hole and attached to a distal end of the bendable body, wherein the intermediate bendable section has a stiffness gradient which differs from a stiffness gradient for the first bendable section.

In another embodiment, the intermediate bendable section of the apparatus comprises of at least two segments, with each segment having a different stiffness gradient.

In further embodiment, the apparatus further comprises a pliable wall extending the length of the bendable body. It is further contemplated that an inside diameter of the at least two guide rings are affixed to at least a portion of the wall. In addition, the wall may further comprise a resilient outer lining for encapsulating the at least two rings.

In other embodiment, the subject apparatus further comprises an actuator attached to a proximal end of the at least one driving wire, wherein the actuator is configured to actuate the driving wire.

In yet additional embodiments, the intermediate bendable section is congruent. In addition, the intermediate bendable section may comprise of at least two adjacent segments, wherein each of the at least two segment have a different stiffness gradient.

In further embodiments, the apparatus comprises a intermediate driving wire slideably situated in the guide hole and attached to the bendably body, wherein the position of attachment for the first and the intermediate driving wires are different along the axial direction of the bendable body.

In yet additional embodiments, the at least two guide holes are configured in each of the at least two guide rings.

It is further contemplated that the driving wire and guide hole may either or both comprise of a radio opaque material.

The subject innovation further teaches a medical apparatus comprising a bendable body having a distal section and a proximal section to be configured to bend with driving wires, the distal section having a skeleton structure comprising; multiple guide rings arrayed with an interval to hold the position of the driving wires, and an inner liner and outer liner attached to the guide rings, and the proximal section comprising: a preferential bending segment having a skeleton structure and connecting to the distal section; a transition skeleton segment comprising of a skeleton structure and connecting to the preferential bending segment; and a passive segment comprising multi-lumen tubing and connecting to the transition skeleton segment, wherein the guide rings, the inner liner and the outer liner form hollow chambers.

In various embodiments, the transition skeleton segment includes a support wire, which is terminated at the distal end of the transition skeleton segment with at the distal end the bending stiffness are increases in the order of the preferential bending segment, the transition skeleton segment, the passive segment.

In other embodiments, the preferential bending segment further includes a second set of the support wires, wherein the transition skeleton segment includes a larger number of the support wires than the preferential bending segment.

In yet another embodiment, the passive segment includes a third set of the support wires situated in the middle of the passive segment.

The subject innovation further teaches a bendable body having a hollow chamber extending the length of the bendable body; a first bendable section; an intermediate bendable section; at least two guide rings disposed in the first bendable section and spaced a distance from one another to create a cavity; at least two guide holes in the bendable body, the guide holes extending the length of the bendable body and parallel with the hollow chamber; and at least one driving wire slideably situated in at least one of the at least two guide hole and attached to a distal end of the bendable body, wherein the intermediate bendable section has a stiffness gradient which differs from a stiffness gradient for the first bendable section.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present invention will become apparent from the following detailed description when taken in conjunction with the accompanying figures showing illustrative embodiments of the present invention.

FIG. 1 is a block diagram of an exemplary bendable medical device incorporating various ancillary components, according to one or more embodiment of the subject apparatus, method or system.

FIG. 2 provides a cut-away perspective view of an exemplary bendable medical device, according to one or more embodiment of the subject apparatus, method or system.

FIG. 3 is a cut-away perspective view of an exemplary bendable medical device, according to one or more embodiment of the subject apparatus, method or system.

FIG. 4a-4d provide a side profile (4a) and front cut-away (4b-4d) views of an exemplary bendable medical device, according to one or more embodiment of the subject apparatus, method or system.

FIG. 5 depicts a side cut-away view of an exemplary bendable medical device, according to one or more embodiment of the subject apparatus, method or system.

FIG. 6 provides a close-up side cut-away view of an exemplary bendable medical device seen in FIG. 5, according to one or more embodiment of the subject apparatus, method or system.

FIGS. 7a-7e provide a side profile (7a) and front cut-away (7b-7d) views of an exemplary bendable medical device, according to one or more embodiment of the subject apparatus, method or system.

FIG. 8 depicts a side cut-away view of an exemplary bendable medical device, according to one or more embodiment of the subject apparatus, method or system.

FIGS. 9a-9e provide a side profile (9a) and front cut-away (9b-9d) views of an exemplary bendable medical device, according to one or more embodiment of the subject apparatus, method or system.

FIGS. 10a-10f provide a side profile (10a) and front cut-away (10b-10f) views of an exemplary bendable medical device, according to one or more embodiment of the subject apparatus, method or system.

FIG. 11 depicts a side cut-away view of an exemplary bendable medical device, according to one or more embodiment of the subject apparatus, method or system.

Throughout the Figures, the same reference numerals and characters, unless otherwise stated, are used to denote like features, elements, components or portions of the illustrated embodiments. In addition, reference numeral(s) including by the designation “′” (e.g. 101′ or 24′) signify intermediateary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

As used herein, the term “substantially” is meant to allow for deviations from the descriptor that do not negatively affect the intended purpose. For example, deviations that are from limitations in measurements, differences within manufacture tolerance, or variations less than 5% can be considered within the scope of substantially the same. The specified descriptor can be absolute value (e.g. substantially spherical, substantially perpendicular, etc.) or relative (e.g. substantially not differing beam waist profile, substantially the same, etc.).

### DETAILED DESCRIPTION OF THE DISCLOSURE

Analysis of the subject innovation has shown that the addition of a section of intermediate stiffness results in a remarkable shift in the occurrence of prolapse, resulting in approximately 30% less failures (where there is no transitional at all). This innovation permits more of the insertion force to be applied to overcome friction elsewhere in the bendable medical device, rather than being limited by the bending stiffness limit of the skeleton section, thus allowing for further advancement of the bendable medical device deeper into corridors of the patient. Furthermore, the innovation allows for improved navigation capabilities by allowing the user to incorporate the new-found flexibility in the bendable medical device to reach portions of the lung faster and with less issues.

For embodiment 1, the advantages of using a skeleton section over the tubing section (flexibility and bending radius) can be maintained while also closing the large stiffness gap.

For embodiment 2, the advantage results in an increase in the stiffness of all skeleton sections to reduce the risk of buckling through the length of the bendable medical device, while also decreasing the relative stiffness differential between the skeleton and tubing section.

In the 3rd embodiment, the advantage results in the ability to create multiple stiffness transitions, which can permit the less-stiff tubing section 115 to bend with a smaller radius due to the short section length.

The subject innovation will now be relayed in detail through the description of the exemplary figures, commencing with the general system associated with the bendable medical device.

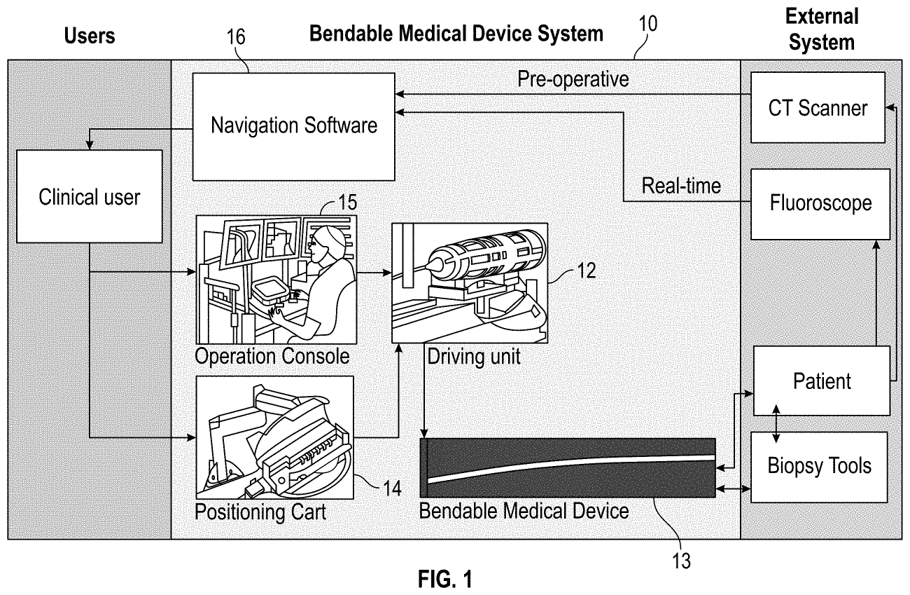

FIG. 1 is a system block diagram of an exemplary bendable medical device system 10 incorporating various ancillary components intended to amass a complete medical system. The bendable medical device system 10 comprises a driving unit 12, a bendable medical device 13, a positioning cart 14, an operation console 15 and navigation software 16. The exemplary bendable medical device system 10 is capable of interacting with external system component and clinical users to facilitate use in a patient.

The navigation software 16 and the driving unit 12 are communicatively-coupled via a bus to transmit/receive data between each other. Moreover, the navigation software 16 is connected and may communicate with a CT scanner, a fluoroscope and an image server (not in Figures), which are ancillary components of the bendable medical device system 10. The image server may include, but is not limited to, a DICOM™ server connected to a medical imaging device including but not limited to a CT and/or MRI scanner and a fluoroscope. The navigation software 16 processes data provided by the driving unit 12 and data provided by images stored on the image server, and/or images from the CT scanner and the fluoroscope in order to display images onto the image display.

The images from the CT scanner may be pre-operatively provided to navigation software 16. With navigation software, a clinical user creates an anatomical computer model from the images. In this particular embodiment, the anatomy is that of a lung with associated airways. From the chest images of the CT scanner, the clinical user can segment the lung airways for clinical treatments, such as biopsy. After generating the lung airway map, the user can also create plan to access the lesion for the biopsy. The plan includes the airways to insert and maneuver the bendable medical device 13 leading to the intended target, which in this example is a lesion.

The driving unit 12 comprises actuators and a control circuitry. The control circuitry is communicatively-coupled with operation console 15. The driving unit 12 is connected to the bendable medical device 13 so that the actuators in the driving unit 12 operate the bendable medical device 13. Therefore, a clinical user can control the bendable medical device 13 via the driving unit 12. The driving unit 12 may also be physically connected to a positioning cart 14. The positioning cart 14 includes a positioning arm, and locates the driving unit 12 and the bendable medical device 13 in the intended position with respect to the target/patient. The clinical user can insert, maneuver and retreat the bendable medical device 13 to perform medical procedures, here a biopsy in the lungs of the patient.

The bendable medical device 13 can be navigated to the target in a patient based on the plan by the clinical user's operation. The bendable medical device 13 includes a tool channel 108 for various tools (e.g. a biopsy tool). The bendable medical device 13 can guide the tool to the lesion of the patient. In one example, the clinical user can take a biopsy sample from the lesion with a biopsy tool.

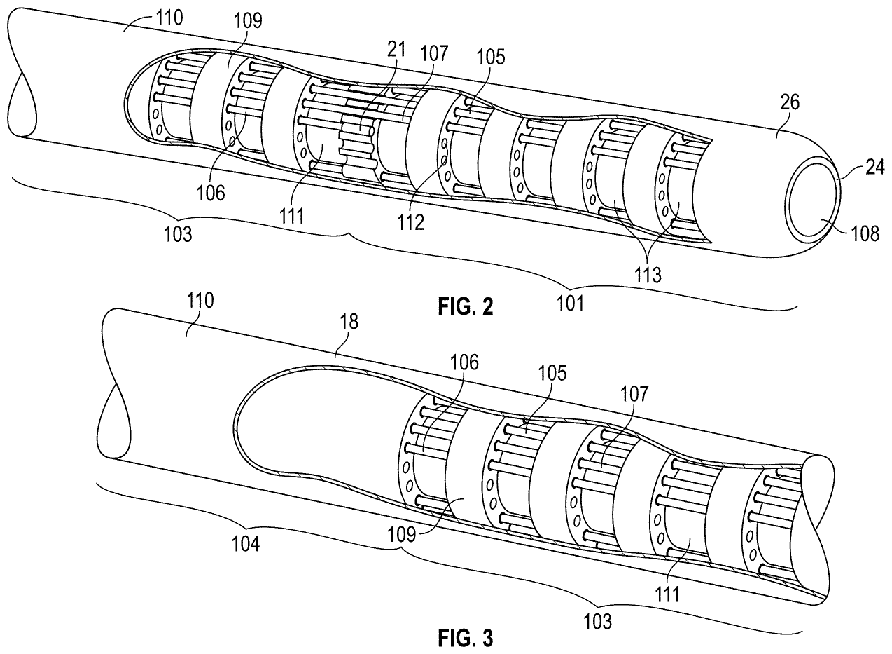

As depicted in FIGS. 2 and 3, the distal section 101 of the bendable medical device 13 comprises multiple guide rings 109, wherein the guide rings 109 are configured a distance apart from one another and do not contact one another. The guide rings 109 are held in place by the cylindrical wall 18, which comprises an inner lining in and an outer lining 110, which provides bendable support to the bendably body 7 while retaining the guide rings 109 in a constant position along the axial direction of the bendable body 17. The inner lining 111 creates an inner diameter 40 and the outer lining 110 creates an outer diameter 42, wherein the inner diameter 40 establishes a tool channel 108. The edge of the bendable body 17 may be rounded by an atraumatic tip 26, to further diminish any harm to the internal elements of a patient as the bendable body 17 is advanced.

The adjacent guide rings 109, are attached to the inner lining 111 and/or outer lining 110, with cavities 113 created between the adjacent guide rings 109, distributed along the longitudinal direction of the bendable body 17. When the bendable body is bent, the cavities 113 create evenly distributed wrinkles in both the inner lining in and outer lining 110. Therefore, the cavities 113 avoid fatal kinking which may crush the tool channel 108.

Each guide ring 109 contains at least two guide holes 112, extending the length of the guide ring 109 parallel with the length of the bendable body 17, for slideable housing of the driving wires 105-106. Furthermore, each guide hole 112 within the guide rings 109, is configured to accept an anchor 21, which is displaced at the end of the driving wires 105-106, to be embedded into the guide ring 109. In FIG. 2, the proximal driving wire 106 depicts the anchor 21, configured at the distal end of the intermediate bendable section 103. The space between adjacent guide rings 109, in cooperation with the resilient inner lining 111 and outer lining 110, allows the bendable body 7 to achieve a greater range of bending motion due to the open space between the guide rings 109, without kinking.

The tool channel 108 is configured to extend the length of the bendable body 17, wherein the proximal end of the bendable body 17 provides access to clinical users for inserting/retreating a medical tool. For example, a clinical user can insert and retrieve a biopsy tool trough the tool channel 108 at the distal end of the bendable medical device 13.

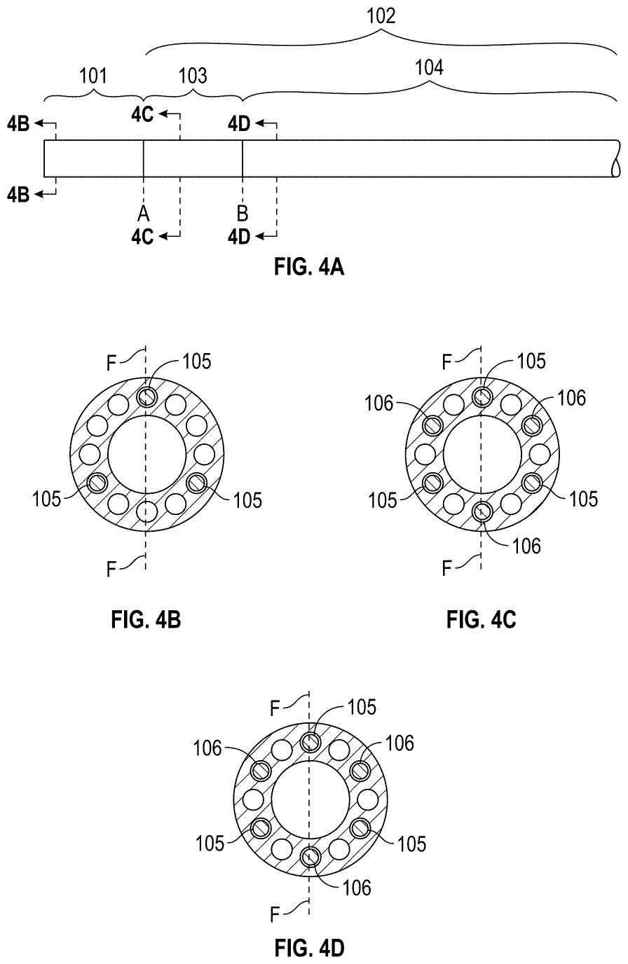

As seen in the cross-sectional views in FIG. 4, the bendable body 17 includes a set of distal driving wires 105, a set of intermediate driving wires 106, and a set of support wires 107, housed in the bendable body 17, wherein each of the set of driving wires 105, and 106, corresponds to the distal, intermediate and proximal bendable sections 101, 103 and 104, respectively. A cylindrical wall 18 is formed by an inner lining in and an outer lining 110 which are congruent and combine with one another at the distal end of the medical device 13 to encapsulate bendable body 17. The wall 18 provides bendable support to the bendably body 17 while retaining the guide rings 109 in a constant position along the axial direction of the bendable body 17. The inner lining 111 creates the inner diameter 40 of the wall and establishes the tool channel 108, while the outer lining 110 creates the outer diameter 42 of the bendable body 17.

The bendable body 17 houses each of the driving wires 105-106 in corresponding guide holes 112, configured along the longitudinal direction of the bendable body 17. The guide holes 112 allow for slideable movement of the driving wires 105-106 along an axial direction of the bendable body 17. The driving wires 105-106 are terminated at the distal end of each bendable section 101, 103 and 104. The distal driving wires 105 are terminated at the distal end of the distal section 101 with anchors 21, and are configured apart from each other by approximately 120 degrees within the bendable body 17. The distal driving wires 105 are connected to the driving unit 12 at the proximal end of the wires 105. The driving unit 12 induces pushing or pulling forces to move the distal driving wires 105 by actuating those wires, and bends the bendable body 17. The proximal driving wires 106 are similarly configured for their corresponding bendable sections 103 and 104, respectively.

Accordingly, by pushing and pulling the driving wires 105 through 106 the proximal, intermediate and the distal bendable sections 104, 103, and 101, respectively, can individually bend the bendable medical device 13, in all three dimensions.

Further depicted in FIG. 2 are support wires 107 provided in the wall 18 of the bendable body 17. The support wires 107 may provide added structural support to the wall 18 and may be anchored to the distal end 24 of a bending sections 101-104. In some embodiments, one or more support wire 107 may be loosely held in the wall 18, allowing for movement and even removal of the support wire 107 to accommodate bending needs in the bendable body 17. The support wires 107 may run through guide holes 112 configured in the wall 18, which may originate at the proximal part of the bendable medical device 13. In certain embodiment, the support wires 107 may be configured for adjustable structural support of the wall 81. Exemplary adjustments for support may include employing various tensile strengths, configurations, resiliency of the support wires 107. In one embodiment, multiple support wires 107 may extend from the distal end 24 of the bendable medical device 13 to the proximal part 105 of the bendable medical device 13, thus allowing all sections 101-104 of the bendable body 17 to gain the kink prevention benefits.

FIG. 4a provides a side view of an exemplary bendable medical device, according to one or more embodiment of the subject innovation, with FIGS. 4b-4d depicting front cross section views of the device in FIG. 4a, at lines C-C, D-D and E-E.

The bendable medical device in FIG. 4a comprises of distal section 101 and proximal section 102 and has a tool channel 108 to deliver a biopsy tools or the other tools through the bendable medical device 13 to the target. The distal driving wires 105 are terminated in the distal end of the distal section 101 and are arrayed equidistantly around the circumference of the device 13 as shown in the cross sectional view of FIG. 4b, in line C-C. In the same manner, proximal driving wires 106 are terminated in the distal end of the proximal section 102 and are also arrayed equidistantly around circumference of the bendable medical device 13 as shown in FIG. 4c, detailing the cross section in line D-D in FIG. 4a.

Also, the proximal driving wires 106 define the proximal end of distal section 101 as position A. By pulling and pushing those wires appropriately, the distal and proximal sections 101 and 102 can bend three-dimensionally. Those sections 101 and 102 can be controlled independently with a robot controller (not shown in the figures). Specifically, the proximal section 102 further comprises of an intermediate bending section 103 and a distal section 104.

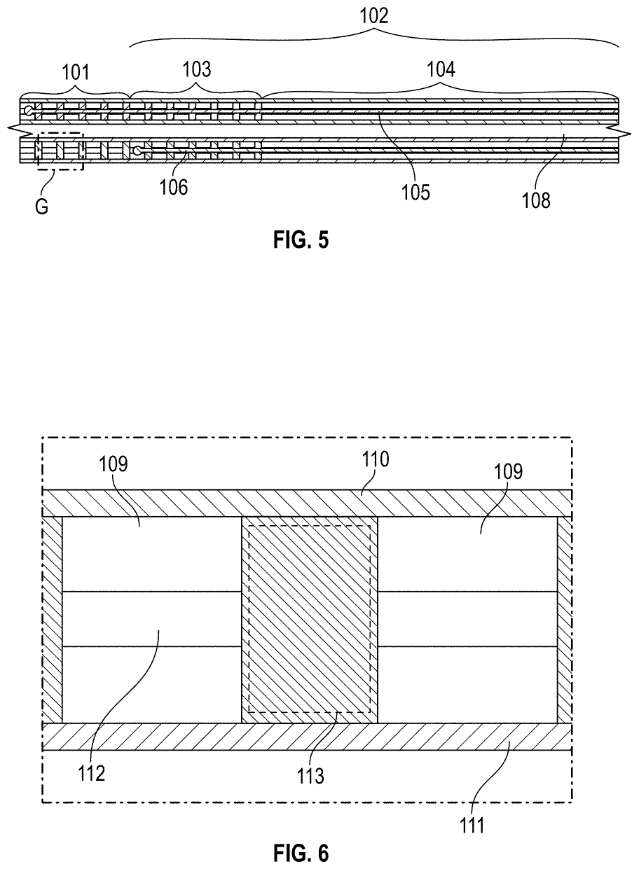

As provided in FIG. 5, the intermediate bending section 103 has the same mechanical structure, named a skeleton structure incorporating multiple guide rings 109 spaced from one another, as the distal section 101, while distal section 104 has a simple multi-guide congruent tubing.

FIG. 6 shows a close-up view of area G in FIG. 5 to better explain the skeleton structure of the distal section 101 and the intermediate bending section 103. The skeleton structure comprises of multiple guide rings 109 with certain intervals between the guide rings 109. The guide rings 109 have guide holes 112 (see FIG. 4b-4d) to slideably hold the driving wires 105 and 106, and are surrounded by an inner liner 110 and an outer liner in. The inner liner 110 creates the tool channel 108, while the outer liner in provide a smooth, continuous surface for optimal insertion into the anatomy and to protect the internal structure of the bendable medical device 13 from external foreign objects. The guide rings 109, inner liner 110 and outer liner in form hollow chambers 113 between respective guide rings 109. These hollow chambers 113 allows the bendable medical device 13 to bend at tight curvatures by promoting small wrinkling and stretching of the inner and outer liners 110 and in into and about hollow chamber 113. In addition, the hollow chamber 113 realizes low bending stiffness compared to the multi-guide congruent tubing section 118. Therefore, in proximal section 102, the intermediate bending section 103 with this skeleton structure has a lower bending stiffness than the distal section 104. This configuration can localize bending into the intermediate bending section 103 in the proximal section 102. When the proximal driving wires 106 are pushed or pulled, the intermediate bending segment 103 primarily bends while the distal section 104 maintains its pose. However, the joint between the intermediate bending segment 103 and the distal section 104 (position B) are subjected to steep transition of bending curvature and tend to suffer from buckling (or kinking). If this buckling occurs, even when the intermediate bending segment 103 turns a bifurcation and reaches the next branch, the distal section 104 following the intermediate bending segment 103 fails to turn the bifurcation and prolapses into the other opening of the bifurcation.

### Embodiment 1

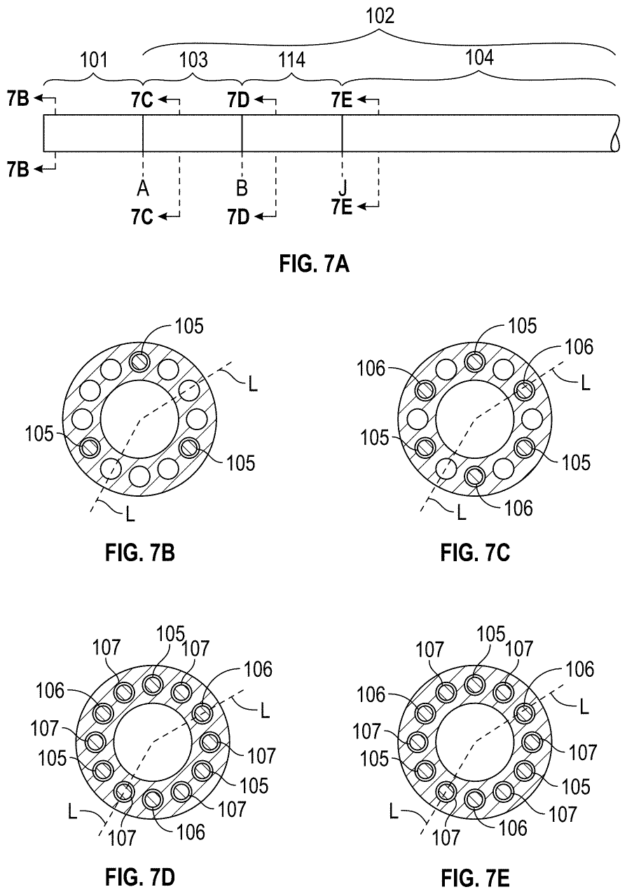

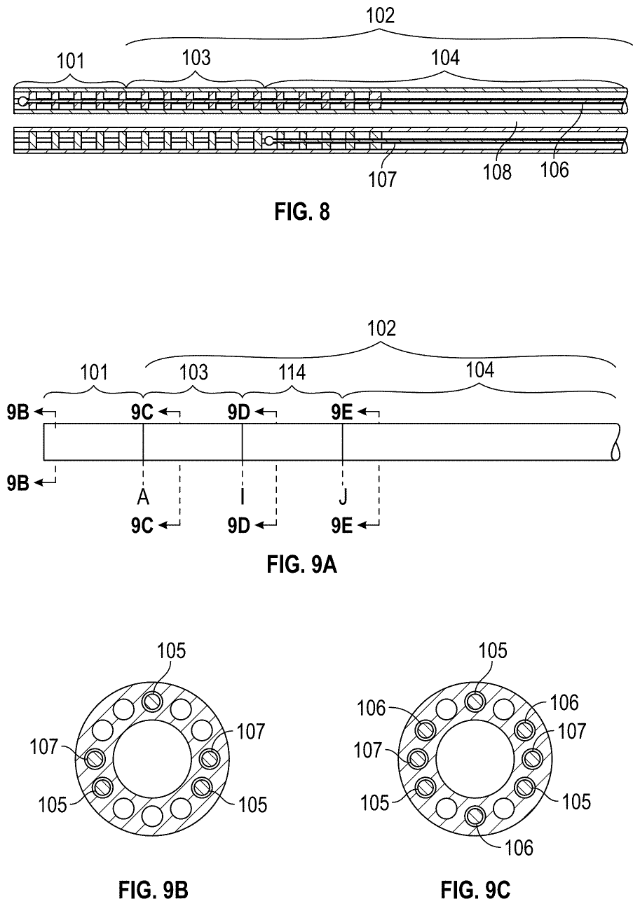

FIG. 7a is a side view of the bendable medical device according to the example detailed in embodiment 1. In addition FIGS. 7b-7e provide cross sectional front views of FIG. 7a, at lines C-C, D-D, H-H and K-K, respectively. Furthermore, FIG. 8 provides a non-uniform side cross sectional view of FIG. 7a, at the L-L line, as seen in FIGS. 7b-7e.

Proximal section 102 now includes a transition skeleton segment 114 between the intermediate bending section 103 and the distal section 104. The transition skeleton segment 114 includes the same skeleton structure as the intermediate bending section 103, and also includes support wires 107. Support wires 107 are terminated at the distal end of the transition skeleton segment 114 (position I in FIG. 7a) and extend through the rest of the bendable medical device 13 toward the proximal end. The proximal end of the support wires 107 are slideable in the bendable medical device 13, and specifically through the guide holes 112.

Support wires 107 provide additional bending stiffness while adjusting for the length change of channels in the bendable medical device 13 when the bendable medical device 13 bends. Since the support wires 107 can be configured between the driving wires 105 and 106, the wall 18 of the bendable medical device 13 can be thinner by eliminating a central backbone structure.

The transition skeleton segment 114 reduces the variance in stiffness between the intermediate bending section 103 and distal section 104 (position B) which significantly mitigates the risk of prolapse in proximal section 102. As the transition in stiffness is diminished, the ‘weak spot’ for inciting prolapse is eliminated, thus yielding a more pliable bendable medical device, with reduced ‘weak spots’ for failure.

### Embodiment 2

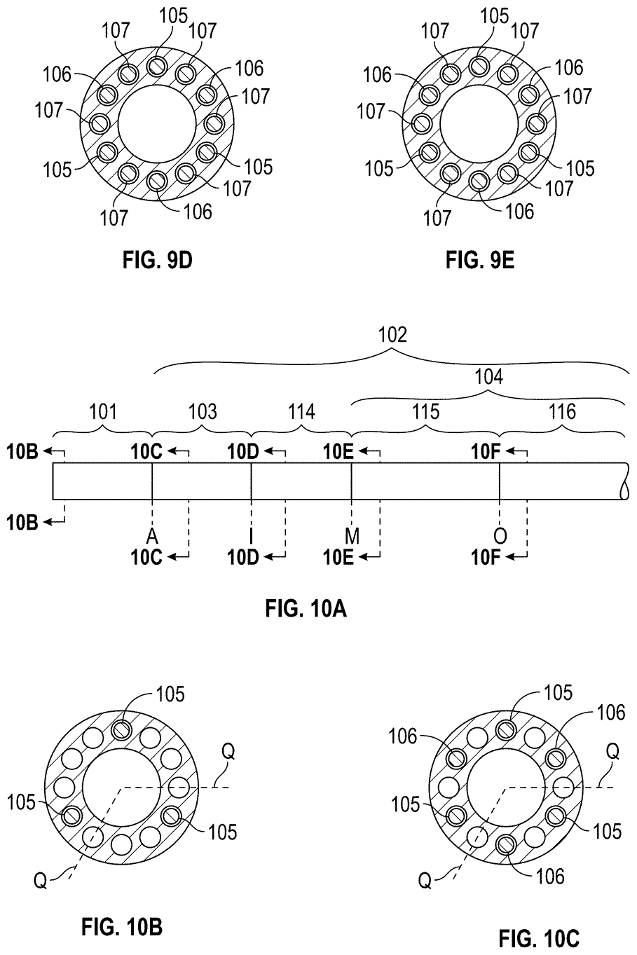

FIG. 9a is a side view of the bendable medical device according to embodiment 2. In addition, FIGS. 9b-9e, provide front cross sectional views of the bendable medical device of FIG. 9a, at lines C-C, D-D, H-H and K-K.

The bendable medical device 13 in this embodiment includes another set of the support wires 107′, which are terminated at the distal end of the distal section 101 and run through the rest of the bendable medical device 13. With the additional set of the support wires 107′, you can increase bending stiffness of distal section 101 and intermediate bending section 103, while maintaining the order of the stiffness. The additional set of the support wires 107′ can also be used to prevent local buckling at the area with the skeleton structure, which are the distal section 101 and intermediate bending section 103. These additional support wires 107′ work in conjunction with the original support wires 107, to increase the stiffness of all sections they traverse. So both the skeleton structure and congruent tubing section 117 will see an equal increase in stiffness. However, this means that the total stiffness difference remains constant, while the relative stiffness difference is reduced.

### Embodiment 3

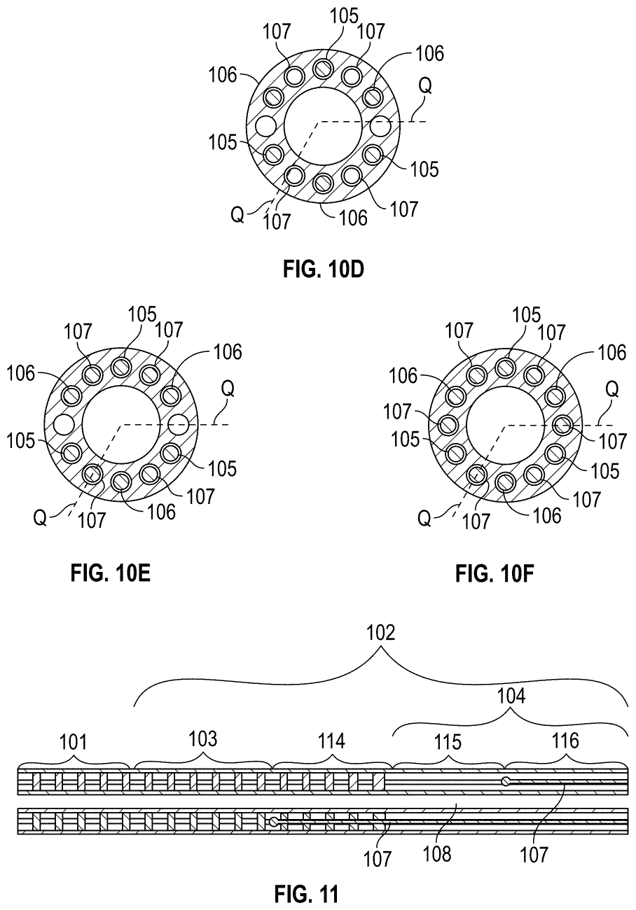

FIGS. 10a-10f and 11 signify another embodiment of the subject innovation, with FIG. boa providing a side view of the bendable medical device in embodiment 3. FIGS. 10b-10f provide us with front cross sectional views of the device in FIG. boa, at lines C-C, D-D, H-H, N-N and P-P.

The difference of this embodiment from embodiment 2 is the starting position of the additional support wires 107′, detailing the flexibility in moving the support wires 107′ to accommodate needs in structural strength.

As seen in the FIGS. 10b-10f, the additional support wires 107′ are terminated in the middle of distal section 104, and create transition tubing segment 115 and passive tubing segment 116. The additional support wires 107′ can add bending stiffness at passive tubing segment 116 and improve push-ability to convey an insertion force to the distal end.

### Design Examples

Two different design variegation's were implemented to address factors contributing to prolapse and failures in the bendable medical device.

The first variation adds a transition portion between sections, here the proximal preferential skeleton section and the proximal passive tubing section, with a stiffness that falls in between the two. The length of this transition portion is variable, and here was chosen to prevent the transition between the proximal skeleton & tubing sections from reaching the Right Upper Lobe (“RUL”).

In one exemplary embodiment or prototype we are incorporating using 40 mm, to make the transition point 90 mm away from the distal end of the bendable medical device. This was chosen based on the current phantoms we are using, where the deepest position of the RUL is less than 90 mm away from the entrance to the RUL. However, the exact length might need to rely on analysis of more patient data or multiple designs can be made and the clinician can be instructed to select a specific design/length based on the patient segmentation.

The intermediate variation also adds a transition section, but reduces the length of the each driven skeleton section to 10 mm each. This will improve the FTL capabilities using the ‘follow-the-leader’ angle based algorithm, since each section will have the same shape as the one that it is following. Improved FTL performance should reduce the occurrence of the bendable medical device getting caught while navigating. The transition section of this variation is longer than the first variation to ensure that the transition point with the passive tubing section is also 90 mm away from the tip of the bendable medical device.

The transition section has the skeleton structure, but the stiffness is increased with the addition of thicker support wires 107 in the 5 remaining unused guide holes 112. The driven skeleton sections already have 3 support wires 107 of 6 thou diameter; the additional support wires 107′ in the transition section have a diameter of 105.5 thou. This diameter wire will fit our bendable medical device design without changing any guide hole or bendable medical device diameters because it is the same diameter as the current driving wires 105 and 106.

There are a number of alternative ways in which this additional stiffness can be achieved, however. In addition to thicker & additional support wires 107, another material can be used altogether, such as stainless steel.

The current implementation has the distal portion of the support wire 107 anchored to the distal portion of the transition section, with the proximal portion running into the tubing section, and are unfixed at the end so they are free to slide with movements in the bendable medical device 13. This is counter-productive, because these support wires will also increase the stiffness of the tubing section, which will negate some the gains received by increasing the stiffness in the skeleton section.

There can also be problems if the support wires end before entering the contiguous section. Since the support wires slide as the bendable medical device bends, there will be shapes in which there is a drop in stiffness at the proximal portion of the transition section because a support wire has slid in the distal direction leaving a gap on the proximal end. At the same time, opposing support wires will have slid into the contiguous section, increasing the stiffness at the distal end of that section. This will create a temporary point of large stiffness differential, which is what we are trying to avoid.

One way to resolve this issue is to have these support wires fixed on both ends. This will create a much larger stiffness addition, which can be desired. Another way to resolve this issue is to have the support wires fixed on the proximal portion of the transition section, and unfixed on the distal side. If we experience the same sliding phenomena as described above, we will not be seeing any large stiffness transitions since the basic transition structure is the same structure as the proximal bending section, so all we will be seeing is a temporary change in the orientation of the transition point rather than adding entirely new stiffness transition points. Since these sections have a skeleton structure, however, there is a risk of the free portion of the driving wire to slide out of a guide wire, and if the bendable medical device it bend it might not re-enter the guide hole it is supposed to be unintentionally dig into other structures of the bendable medical device, potentially damaging it. This can be mitigated by running a low stiffness (or high stiffness, if desired) liner in between all of the guide rings where this issue might occur.

Instead of moving the proximal end point of these support wires distally, they can also be run all the way to the hub and fixed in various ways. One way is to fixed it to a spring or other type of surface that can be moved with a specific, or varying, amount of force. Alternatively, it can be fixed in a way that can be toggled between ‘free to slide’/‘slide with resistance’/‘completely fixed’. One way this can be one is with a set screw that can be screwed in/out to change the resistance. Similarly, this end can be fixed to a motor which can toggle between fixed/free as well as have a force feedback system to adjust the resistance.

To further expand on the liner concept, a liner or hypotube can also be placed along guide holes that are currently occupied by other support wires or driving wires, remove the restriction of only using empty guide holes.

In addition to placing material inside the guide holes of the bendable medical device, additional inner/outer covers can be applied to the section to increase the stiffness. The material and thickness of these covers can vary to reach the desired stiffness. Alternatively, if a change in OD/ID is not desired, the pre-existing inner/outer cover for the skeleton structure can be replaced in this transition section with a different material, or with a different thickness (the guide rings can also modified with a different ID/OD to ensure the final diameters do not change). This additional or supplementary material can also have a stiffness transition along its length as well, such as a variable pitch spring.

Another way to implement a stiffness gradient is to adjust the spacing of the guide rings. This can be done gradually or discretely. Alternatively, additional material can be placed in between the guide rings, and the stiffness of this material can vary gradually or discretely along the length of the section, based on the material selection or thickness.

Instead of using the skeleton structure, variations to the tubing section can be used. A different material with a lower stiffness can be used and welded to the 40D section of the tubing section. Alternatively, instead of welding different materials together, additional inner/outer/intermediate materials can be added to create a composite and increase the stiffness along the proximal direction. Another way to adjust the stiffness is to have the diameter of the bendable medical device can be gradually, or discretely, increased. One way to achieve this is with a bump extrusion. Or, additional support wires (with the various alternatives mentioned above) can be placed at different points along the length of the tubing section.

1. A medical apparatus comprising:
a bendable body having a hollow chamber extending the length of the bendable body;
a distal bendable section;
an intermediate bendable section;
at least two guide rings disposed in the distal bendable section and spaced a distance from one another to create a cavity;
at least two guide holes in the bendable body, the guide holes extending the length of the bendable body and configured about and parallel to the hollow chamber; and
at least one driving wire slideably situated in at least one of the at least two guide hole and attached to a distal end of the bendable body,
wherein the intermediate bendable section has a stiffness gradient that differs from a stiffness gradient for the distal bendable section.

2. The apparatus of claim 1, wherein the intermediate bendable section comprises of at least two segments, with each segment having a different stiffness gradient.

3. The apparatus of claim 1, further comprising a pliable wall extending the length of the bendable body.

4. The apparatus of claim 3, wherein an inside diameter of the at least two guide rings are affixed to at least a portion of the wall.

5. The apparatus of claim 3, wherein the wall further comprises a resilient outer lining for encapsulating the at least two rings.

6. The apparatus of claim 1, further comprising an actuator attached to a proximal end of the at least one driving wire, wherein the actuator is configured to actuate the driving wire.

7. The apparatus of claim 1, wherein the intermediate bendable section is congruent.

8. The apparatus of claim 7, wherein the intermediate bendable section comprises of at least two adjacent segments, wherein each of the at least two segment have a different stiffness gradient.

9. The apparatus of claim 1, further comprising a intermediate driving wire slideably situated in the guide hole and attached to the bendably body, wherein the position of attachment for the first and the intermediate driving wires are different along the axial direction of the bendable body.

10. The apparatus of claim 1, wherein the first and intermediate guide holes are configured in each of the at least two guide rings.

11. The apparatus of claim 1, wherein the driving wire and guide hole comprise of a radio opaque material.

12. A medical apparatus comprising a bendable body having a hollow chamber extending the length of the bendable body, the bendable body comprising:
a first bendable section at the distal end of the bendable body;
a intermediate bendable section proximal to the first bendable section;
a first guide hole in the bendable body, the first guide hole being parallel with the hollow chamber;
a distal driving wire slideably situated in the first guide hole and attached to a distal end of the first bendable section;
an intermediate guide hole in the bendable body, the intermediate guide hole being parallel with
the hollow chamber; and
a intermediate driving wire slideably situated in the intermediate guide hole and attached to
a distal end of the intermediate bendable section,
wherein the first bendable section comprises a flexible structure, and
wherein a stiffness at the distal end of the intermediate bendable section is substantially the same as a stiffness of the first bendable section, and less than the stiffness at a proximal end of the intermediate bendable section.

13. The apparatus of claim 12, further comprising a pliable wall extending the length of the bendable body.

14. The apparatus of claim 12, wherein the bendable body comprises of at least two guide rings disposed in the first bendable section and spaced a distance from one another to create a cavity between the at least two guide rings.

15. The apparatus of claim 14, wherein the first and intermediate guide holes are configured in each of the at least two guide rings.

16. The apparatus of claim 12, further comprising an actuator attached to a proximal end of the distal driving wire, wherein the actuator is configured to actuate the driving wire.

17. The apparatus of claim 12, further comprising an actuator attached to a proximal end of the intermediate driving wire, wherein the actuator is configured to actuate the driving wire.

18. The apparatus of claim 12, wherein the driving wire and guide hole comprise of a radio opaque material.
